(19) United States
(12) Patent Application Publication
(10) Pub. No.: US 2007/0233250 A1
(43) Pub. Date: Oct. 4, 2007

(54) SYSTEMS FOR TREATING BONE

(76) Inventor: John H. Shadduck, Tiburon, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 11/672,434

(22) Filed: Feb. 7, 2007

Related U.S. Application Data
(60) Provisional application No. 60/765,852, filed on Feb. 7, 2006.

(51) Int. Cl. A61F 2/44

(52) U.S. Cl. 623/17.11

(57) ABSTRACT

The present invention relates in certain embodiments to medical devices for treating vertebral compression fractures. In one embodiment, the invention relate to systems for introducing fill material into a vertebral body that slowly expands vertebral height without explosive balloon expansion as in kyphoplasty. The system provides a fill material that infills a vertebra without flowable bone cement as used in kyphoplasty and vertebropalsty procedures. Thus, the bone fill system prevents the possibility of extravasation of material into the spinal canal which occurs in a significant number of kyphoplasty and vertebropalsty procedures. An energy source can apply energy to a substantially rigid implant material in order to soften, melt, or fracture the implant material to infill a vertebral body.

### CROSS-REFERENCE TO RELATED APPLICATIONS

This application claims the benefit under 35 U.S.C. § 119(e) of U.S. Provisional Patent Application No. 60/765,852, filed on Feb. 7, 2006. This application is also related to U.S. patent application Ser. No. 11/165,652, filed Jun. 24, 2005, now U.S. Pub. No. 2006-0122623; U.S. patent application Ser. No. 11/165,651, filed Jun. 24, 2005, now U.S. Pub. No. 2006-0122622; U.S. patent application Ser. No. 11/208,448, filed Aug. 20, 2005, now U.S. Pub. No. 2006-0122621; U.S. patent application Ser. No. 11/469,764, filed Sep. 1, 2006; U.S. application Ser. No. 11/209,035, filed Aug. 22, 2005, now U.S. Pub. No. 2006-0122625; U.S. application Ser. No. 11/196,045, filed Aug. 2, 2005, now U.S. Pub. No. 2006-0122624; and U.S. application Ser. No. ___/______, filed Feb. 7, 2007 (Atty. Docket No. DFINE.031A) and titled “METHODS FOR TREATING BONE.” The entire contents of all of the above applications are hereby incorporated by reference and should be considered a part of this specification.

### BACKGROUND OF THE INVENTION

1. Field of the Invention

The present invention relates in certain embodiments to medical devices for osteoplasty procedures such as treating vertebral compression fractures. More particularly, embodiments of the invention relate to instruments and systems for introducing fill material into a vertebral body that (i) slowly expands vertebral height without explosive balloon expansion as in kyphoplasty, and (ii) that infills a vertebra without flowable material as in kyphoplasty and vertebropalsty wherein bone cement can result in extravasation into the spinal canal.

2. Description of the Related Art

Osteoporotic fractures are prevalent in the elderly, with an annual estimate of 1.5 million fractures in the United States alone. These include 750,000 vertebral compression fractures (VCFs) and 250,000 hip fractures. The annual cost of osteoporotic fractures in the United States has been estimated at $13.8 billion. The prevalence of VCFs in women age 50 and older has been estimated at 26%. The prevalence increases with age, reaching 40% among 80-year-old women. Medical advances aimed at slowing or arresting bone loss from aging have not provided solutions to this problem. Further, the population affected will grow steadily as life expectancy increases. Osteoporosis affects the entire skeleton but most commonly causes fractures in the spine and hip. Spinal or vertebral fractures also cause other serious side effects, with patients suffering from loss of height, deformity and persistent pain which can significantly impair mobility and quality of life. Fracture pain usually lasts 4 to 6 weeks, with intense pain at the fracture site. Chronic pain often occurs when one vertebral level is greatly collapsed or multiple levels are collapsed.

Postmenopausal women are predisposed to fractures, such as in the vertebrae, due to a decrease in bone mineral density that accompanies postmenopausal osteoporosis. Osteoporosis is a pathologic state that literally means “porous bones”. Skeletal bones are made up of a thick cortical shell and a strong inner meshwork, or cancellous bone, of with collagen, calcium salts and other minerals. Cancellous bone is similar to a honeycomb, with blood vessels and bone marrow in the spaces. Osteoporosis describes a condition of decreased bone mass that leads to fragile bones which are at an increased risk for fractures. In an osteoporosis bone, the sponge-like cancellous bone has pores or voids that increase in dimension making the bone very fragile. In young, healthy bone tissue, bone breakdown occurs continually as the result of osteoclast activity, but the breakdown is balanced by new bone formation by osteoblasts. In an elderly patient, bone resorption can surpass bone formation thus resulting in deterioration of bone density. Osteoporosis occurs largely without symptoms until a fracture occurs.

Vertebroplasty and kyphoplasty are recently developed techniques for treating vertebral compression fractures. Percutaneous vertebroplasty was first reported by a French group in 1987 for the treatment of painful hemangiomas. In the 1990's, percutaneous vertebroplasty was extended to indications including osteoporotic vertebral compression fractures, traumatic compression fractures, and painful vertebral metastasis. Vertebroplasty is the percutaneous injection of PMMA (polymethylmethacrylate) into a fractured vertebral body via a trocar and cannula. The targeted vertebrae are identified under fluoroscopy. A needle is introduced into the vertebrae body under fluoroscopic control, to allow direct visualization. A bilateral transpedicular (through the pedicle of the vertebrae) approach is typical but the procedure can be done unilaterally. The bilateral transpedicular approach allows for more uniform PMMA infill of the vertebra.

In a bilateral approach, approximately 1 to 4 ml of PMMA is used on each side of the vertebra. Since the PMMA needs to be is forced into the cancellous bone, the techniques require high pressures and fairly low viscosity cement. Since the cortical bone of the targeted vertebra may have a recent fracture, there is the potential of PMMA leakage. The PMMA cement contains radiopaque materials so that when injected under live fluoroscopy, cement localization and leakage can be observed. The visualization of PMMA injection and extravasation are critical to the technique—and the physician terminates PMMA injection when leakage is evident. The cement is injected using syringes to allow the physician manual control of injection pressure.

Kyphoplasty is a modification of percutaneous vertebroplasty. Kyphoplasty involves a preliminary step consisting of the percutaneous placement of an inflatable balloon tamp in the vertebral body. Inflation of the balloon creates a cavity in the bone prior to cement injection. The proponents of percutaneous kyphoplasty have suggested that high pressure balloon-tamp inflation can at least partially restore vertebral body height. In kyphoplasty, some physicians state that PMMA can be injected at a lower pressure into the collapsed vertebra since a cavity exists, when compared to conventional vertebroplasty.

The principal indications for any form of vertebroplasty are osteoporotic vertebral collapse with debilitating pain. Radiography and computed tomography must be performed in the days preceding treatment to determine the extent of vertebral collapse, the presence of epidural or foraminal stenosis caused by bone fragment retropulsion, the presence of cortical destruction or fracture and the visibility and degree of involvement of the pedicles.

Leakage of PMMA during vertebroplasty can result in very serious complications including compression of adjacent structures that necessitate emergency decompressive surgery. See “Anatomical and Pathological Considerations in Percutaneous Vertebroplasty and Kyphoplasty: A Reappraisal of the Vertebral Venous System”, Groen, R. et al, Spine Vol. 29, No. 13, pp 1465-1471 2004. Leakage or extravasation of PMMA is a critical issue and can be divided into paravertebral leakage, venous infiltration, epidural leakage and intradiscal leakage. The exothermic reaction of PMMA carries potential catastrophic consequences if thermal damage were to extend to the dural sac, cord, and nerve roots. Surgical evacuation of leaked cement in the spinal canal has been reported. It has been found that leakage of PMMA is related to various clinical factors such as the vertebral compression pattern, and the extent of the cortical fracture, bone mineral density, the interval from injury to operation, the amount of PMMA injected and the location of the injector tip. In one recent study, close to 50% of vertebroplasty cases resulted in leakage of PMMA from the vertebral bodies. See Hyun-Woo Do et al, “The Analysis of Polymethylmethacrylate Leakage after Vertebroplasty for Vertebral Body Compression Fractures”, Jour. of Korean Neurosurg. Soc. Vol. 35, No. 5 (5/2004) pp. 478-82, (http://www jkns.or.kr/htm/abstract.asp?no=0042004086).

Another recent study was directed to the incidence of new VCFs adjacent to the vertebral bodies that were initially treated. Vertebroplasty patients often return with new pain caused by a new vertebral body fracture. Leakage of cement into an adjacent disc space during vertebroplasty increases the risk of a new fracture of adjacent vertebral bodies. See Am. J. Neuroradiol. 2004 February; 25(2):175-80. The study found that 58% of vertebral bodies adjacent to a disc with cement leakage fractured during the follow-up period compared with 12% of vertebral bodies adjacent to a disc without cement leakage.

Another life-threatening complication of vertebroplasty is pulmonary embolism. See Bernhard, J. et al, “Asymptomatic diffuse pulmonary embolism caused by acrylic cement: an unusual complication of percutaneous vertebroplasty”, Ann. Rheum. Dis. 2003;62:85-86. The vapors from PMMA preparation and injection also are cause for concern. See Kirby, B, et al., “Acute bronchospasm due to exposure to polymethylmethacrylate vapors during percutaneous vertebroplasty”, Am. J. Roentgenol. 2003; 180:543-544.

In both higher pressure cement injection (vertebroplasty) and balloon-tamped cementing procedures (kyphoplasty), the methods do not provide for well controlled augmentation of vertebral body height. The direct injection of bone cement simply follows the path of least resistance within the fractured bone. The expansion of a balloon applies also compacting forces along lines of least resistance in the collapsed cancellous bone. Thus, the reduction of a vertebral compression fracture is not optimized or controlled in high pressure balloons as forces of balloon expansion occur in multiple directions.

In a kyphoplasty procedure, the physician often uses very high pressures (e.g., up to 200 or 300 psi) to inflate the balloon which crushes and compacts cancellous bone. Expansion of the balloon under high pressures close to cortical bone can fracture the cortical bone, typically the endplates, which can cause regional damage to the cortical bone with the risk of cortical bone necrosis. Such cortical bone damage is highly undesirable as the endplate and adjacent structures provide nutrients for the disc.

Kyphoplasty also is problematic in that balloon inflation expansion does not slowly expand to displace and compact cancellous bone. Instead, the balloon in restrained in a substantially compacted shape until the inflation forces overcome the resistance of the ceramic-like cancellous bone at which time the balloon explosively expands. Such explosive expansion of the balloon can cause fat in the bone marrow as well as blood to be displaced into the venous system—wherein the fat can result in dangerous emboli.

There is a general need to provide bone cements and methods for use in treatment of vertebral compression fractures that provide a greater degree of control over introduction of cement and that provide better outcomes. The present invention meets this need and provides several other advantages in a novel and nonobvious manner.

### SUMMARY OF THE INVENTION

Certain embodiments disclosed herein provide vertebroplasty systems for infill of abnormal bone without the possibility of extravasation. One embodiment comprises an implant body that is elongated and substantially strong to allow the body to be driven axially or helically through an introducer into bone. At the distal end of the introducer, the elongated implant yields to a thermal mechanical energy to transform into a non-elongated configuration for filling a controlled geometry in the bone.

In accordance with one embodiment, a system for treating bone is provided. The system comprises an elongated introducer configured for insertion in a bone, the introducer defining a passage extending therethrough along an axis of the introducer, and an elongated implant body configured for insertion through the passage into the bone, the elongated implant body having a substantially unyielding configuration. The system also comprises an energy source coupled to the elongated introducer, the energy source configured to transform at least a portion of the elongated implant into a yielding configuration to infill the bone.

In accordance with another embodiment, a system for treating a vertebra is provided, comprising an elongated introducer configured for insertion in a vertebral body, the introducer defining a passage extending therethrough along an axis of the introducer. The system also comprises a substantially rigid implant configured for insertion through the passage into the vertebral body, the implant comprising a plurality of implant elements coupled with each other via a junction, the junction transformable between a substantially unyielding configuration to a substantially yielding configuration. The system further comprises an energy source coupled to the elongated introducer, the energy source configured to separate at least one implant element from the implant at said junction, the separated implant element advanced into the vertebral body to infill the vertebral body.

In accordance with still another embodiment, a system for the infill of the interior of a bone is provided. The system comprises an elongated introducer configured for introduction into a bone, the introducer defining a passage extending therethrough along an axis of the introducer. The system also comprises a plurality of implants dimensioned for advancement along the introducer, each adjacent pair of the plurality of implants coupled to each other via a junction, each implant having a first helical feature for cooperating with a second helical feature on the introducer to helically advance the implants through the passage and into the bone.

In accordance with yet another embodiment, a system for treating a bone is provided. The system comprises an elongated introducer configured for insertion into a bone, the introducer defining a passage extending therethrough along an axis of the introducer. The system also comprises an elongated implant body configured for insertion into the bone along said introducer, the elongated implant body having a substantially unyielding configuration, a surface of the implant body configured to engage with a surface of the introducer for advancement of the implant body along the introducer. The system further comprises means for transforming at least a portion of the elongated implant from a substantially unyielding configuration to a yielding configuration to infill the bone.

In accordance with another embodiment, an implant configured for insertion into a bone is provided. The implant comprises an elongated body sized for introduction along an introducer into a bone, at least a portion of the elongated body being transformable from a substantially unyielding configuration to a yielding configuration configured for infilling the bone.

In accordance with still another embodiment, an implant configured for insertion into a bone is provided, comprising an elongated body having at least one helical feature and sized for introduction into a bone, at least a portion of the elongated body being severable from the elongated portion for infilling the bone.

In accordance with one embodiment, a method for treating a vertebral body is provided. The method comprises advancing an elongated implant body through an introducer and into an interior of a vertebral body, the elongated implant body having a first configuration that is substantially unyielding along a longitudinal axis of the body. The method also comprises transforming the implant body to a second configuration that yields its first configuration proximate to a working end of the introducer for infilling a region of the vertebral body.

In accordance with another embodiment, a method for treating a bone is provided. The method comprises inserting an introducer into a bone, helically driving an elongated implant body relative to the introducer, the implant body having a first elongated configuration, and transforming at least a portion of the implant body into a second non-elongated configuration for infilling a region of the bone.

In accordance with still another embodiment, a method for treating a vertebra is provided. The method comprises positioning a distal end of an introducer in an interior of a vertebra, and helically driving at least one implant body comprising a helical feature through the introducer to thereby deploy the implant body in the interior of the vertebra, the helical feature engaging a cooperating feature on the introducer.

### BRIEF DESCRIPTION OF THE DRAWINGS

In order to better understand the invention and to see how it may be carried out in practice, some preferred embodiments are next described, by way of non-limiting examples only, with reference to the accompanying drawings, in which like reference characters denote corresponding features consistently throughout similar embodiments in the attached drawings.

FIG. 1A is a schematic perspective view of spine segment showing an introducer in a pedicular access.

FIG. 1B is a schematic view of the spine segment of FIG. 1A from a different angle.

FIG. 2A is a perspective schematic view of a bone implant and introducer in accordance with one embodiment.

FIG. 2B is a perspective schematic view of a bone implant and introducer in accordance with another embodiment.

FIG. 2C is a perspective schematic view of a bone implant and introducer in accordance with yet another embodiment.

FIG. 3 is a schematic view of the implant and introducer of FIG. 2 in a method using thermal energy to alter a property of a polymer implant, in accordance with one embodiment.

FIG. 4 is a plan schematic view of another implant body with helical features for cooperating with a threaded introducer, in accordance with another embodiment.

FIG. 5 is a schematic view of the implant body and introducer of FIG. 4 in a method using Rf energy to alter a property of a polymer implant being used in a vertebral body treatment, in accordance with one embodiment.

FIG. 6 is a plan schematic view of another implant body with helical features for cooperating with a threaded introducer similar to that of FIG. 4 with spaced apart fracturable portions.

FIG. 7 is a sectional schematic view of another implant body with helical features in the interior of the body and polygonal features on the exterior surface of the body with spaced apart fracturable portions.

FIG. 8 is a sectional schematic view of a portion of another implant body with helical features and regions that are adapted to be cut at a distal end of an introducer.

FIG. 9 is a plan schematic view of another implant system comprising a plurality of elements with helical features for cooperating with a threaded introducer.

FIG. 10 is a schematic view of the implant elements and introducer of FIG. 9 in a method of infilling a vertebral body.

FIG. 11 is a schematic view of another form of implant elements with helical features in an interior bore for use in infilling a vertebral body.

### DETAILED DESCRIPTION OF THE PREFERRED EMBODIMENTS

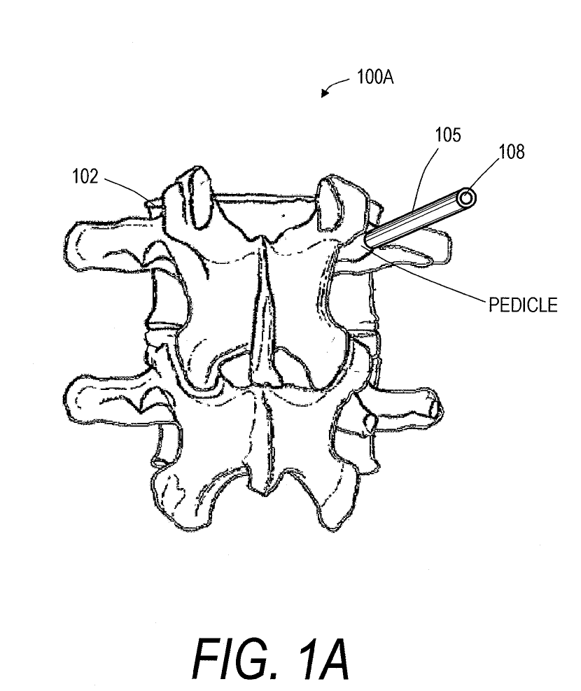

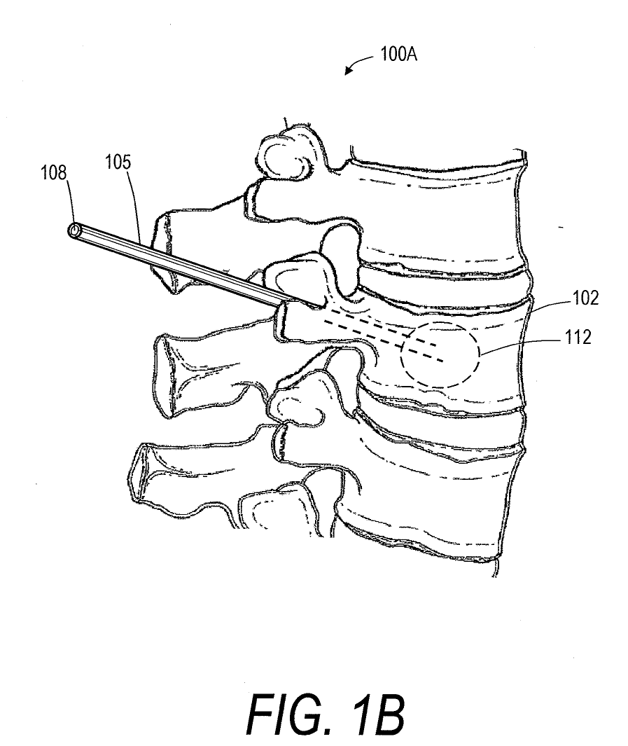

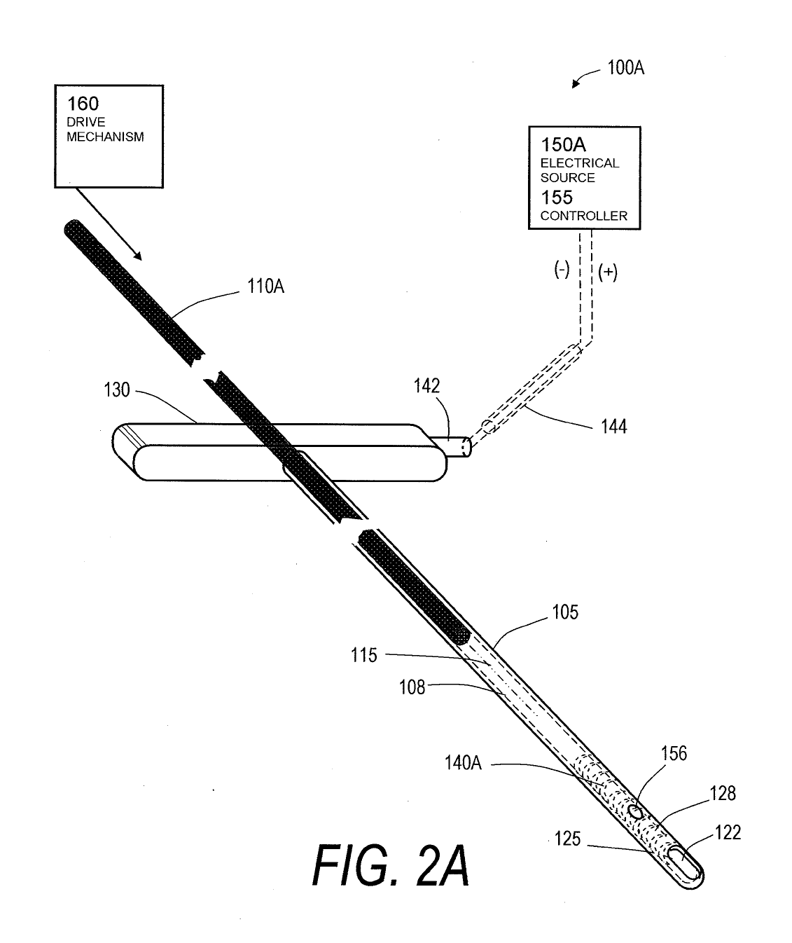

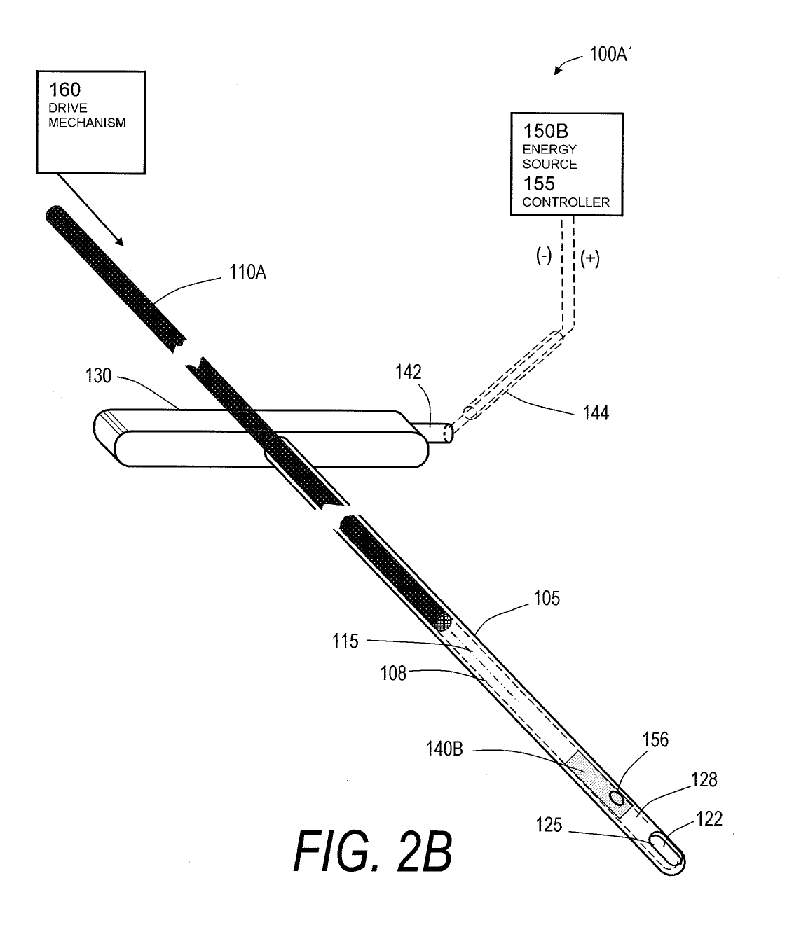

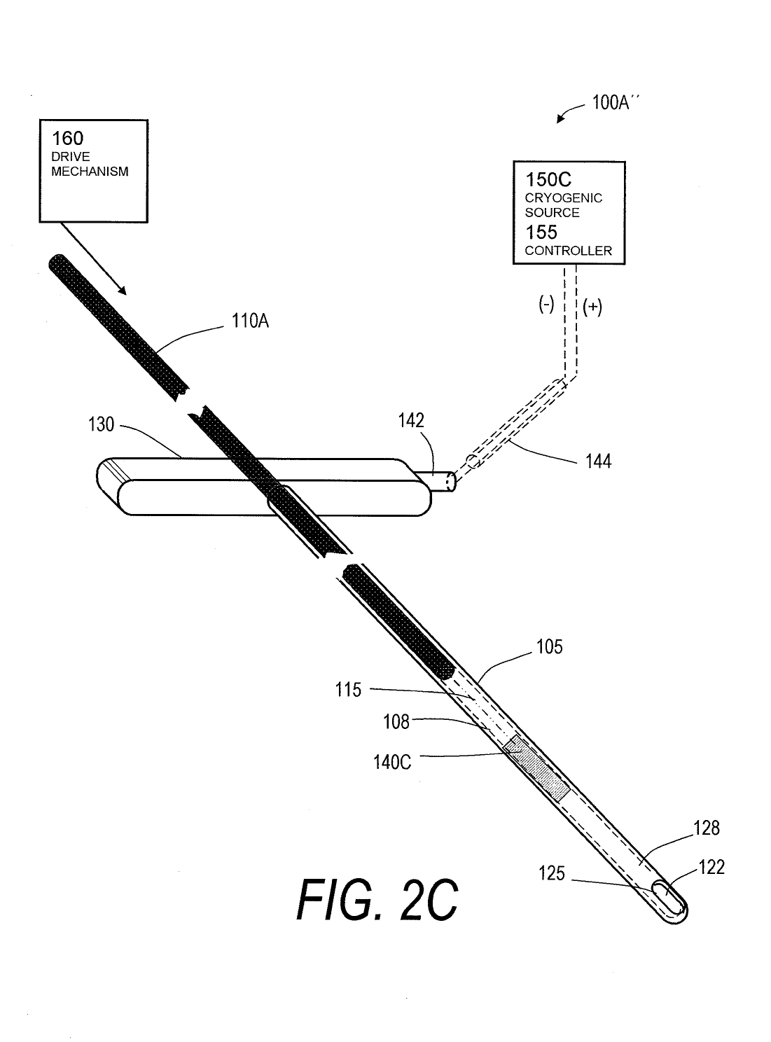

Referring to FIGS. 1A and 1B, one embodiment of bone fill introducer or injector system 100A is shown that is configured for treatment of an abnormal vertebra 102 such as in the case of a vertebral compression fracture. Introducer system 100A as in FIGS. 2A-C includes introducer sleeve 105 with passageway 108 therein that is configured for the introduction of an elongated implant body 110A therethrough to a targeted site 112 in a vertebra (see FIGS. 1A-1B). As can be seen in FIG. 2, the elongated implant body 110A has a first configuration that is substantially unyielding along a longitudinal axis 115 of the body. The term “unyielding” as used herein means that the implant body is substantially rigid, inflexible and sufficiently strong to allow the implant to be axially pushed or driven through passageway 108 in the introducer sleeve 105. The implant body is preferably of a polymeric material, a ceramic material, a glass material or a combination thereof that allows transformation of the implant to a “yielding” material at the working end 120 of the introducer 105 for filling the targeted site. The term “yielding” is used to describe that implant as having a softened, melted, fractured, cut, partially sacrificed or other “yielded” or “yielding” configuration that will be described in more detail below.

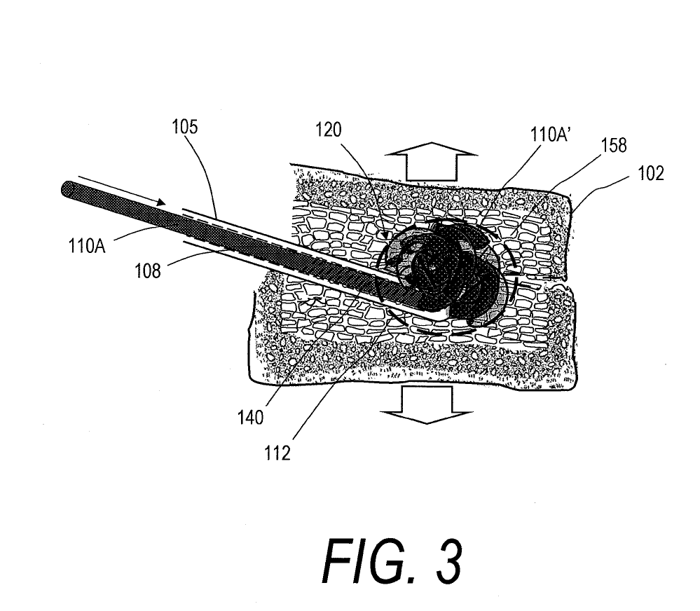

One embodiment of a method for treating a vertebra includes providing the implant body described above, advancing the implant body 110A through the introducer passageway 108 or channel to exit the open end or outlet 122 thereof into an interior of a vertebra, and transforming the implant body to a second configuration 110A′ (FIG. 3) that yields its first configuration proximate to working end 120 of sleeve 105 to thereby allow infilling a site 112 in the vertebra wherein the implant forms a more or less compacted form and geometry (see FIG. 3), rather than an elongated geometry as when the implant is inserted into the proximal or handle end 130 of sleeve 105. As can be seen in FIGS. 2A-C, the introducer has a side-directed outlet 122 in the working end but the outlet also can be distally oriented as the end of a needle.

In one embodiment, still referring to FIGS. 2A-C, the means for transforming the implant from the substantially rigid or unyielding configuration of FIGS. 2A-C to the softened, yielding configuration of FIG. 3 comprises a thermal energy deliver means or emitter in the form of resistively heated coil emitter or a resistively heated positive temperature coefficient of resistance (PTCR) emitter 140A, 140B or 140C in bore 108 in the working end 120. In FIG. 2A, it can be seen that handle 130 coupled to introducer sleeve 105 includes an electrical connector 142 for coupling electrical source 150A to the connector by means of electrical cable 152. The system further includes a controller 155 for controlling electrical energy delivery to the coil or PTCR emitter 140A. In one embodiment, the working end 120 carries a thermocouple 156 proximate to the coil or PTCR emitter 140A or electrode that is operatively coupled to the controller for modulating energy delivery to the emitter to thereby control heating of the implant body 110A. In operation, (i) the implant 110A is introduced through the sleeve 105, (ii) the emitter 140A is contemporaneously actuated in distal portion of the introducer bore, and (iii) the implant body is pushed into cancellous bone 158 wherein the softened implant body 110A′ becomes a convoluted mass and thus applies height restoring forces on the VCF. As can be seen in FIG. 2A, the drive mechanism indicated at 160 can be any means of applying force such as a human hand, a mechanical assist drive system such as a gear which cooperates with surface features on the implant body, a hydraulic assist drive system, a helical drive system (as described below) or the like.

The implant body 110A can be any form of biocompatible polymer such as a PMMA that is softenable or meltable by heating. The implant system can further include the introduction of a hardenable bone cement together with implant body 110A by another inflow channel in the introducer. Alternatively, the implant body can be configured with surfaces that fuse together upon heating to provide higher strength in the convoluted form (FIG. 3).

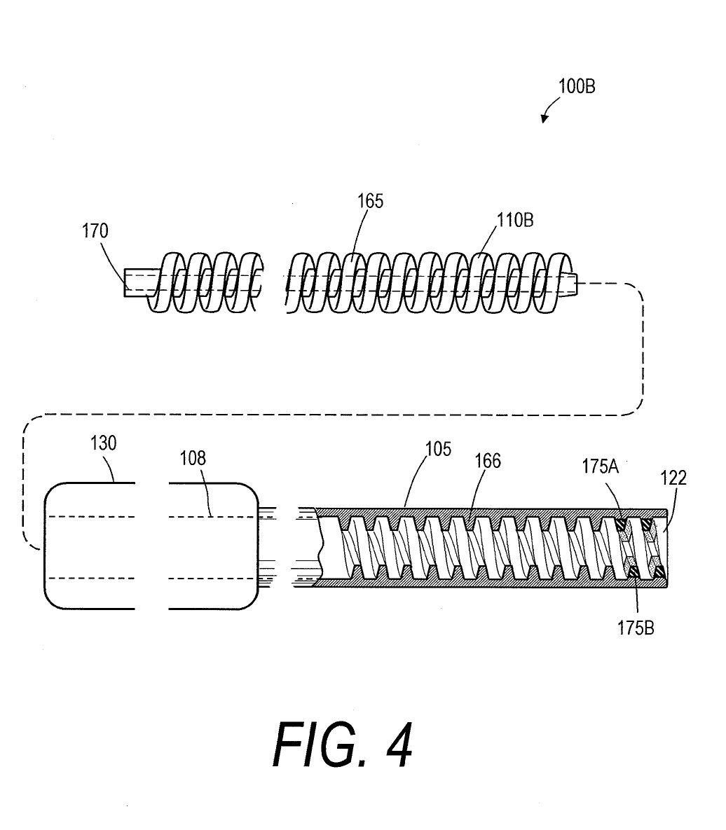

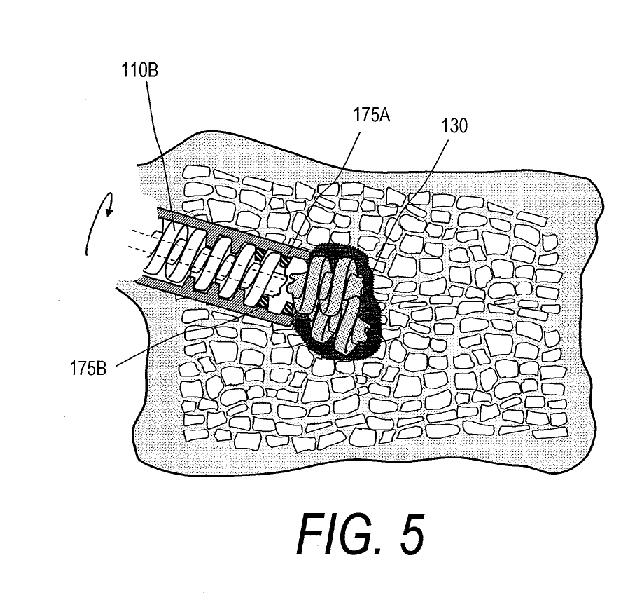

FIGS. 4 and 5 illustrate another system embodiment 110B with the implant body 110B having helical surface features indicated as threads 165 that cooperate with threaded features 166 in at least a portion of bore 108 in sleeve 105. A motor drive or hand drive can rotate an elongated polygonal shaft that is configured to mate with polygonal (hex) bore 170 in the implant body for driving the implant. In the embodiment of FIGS. 4 and 5, the thermal energy emitter comprises opposing polarity electrodes 175A and 175B that carry Rf energy to an electrically conductive implant body 110B. For example, the polymeric implant can be conductively doped with carbon, a metal or the like in the form of particles, filaments or the like. In use, referring to FIG. 5, the system can be used with high energy densities to cause a fuse-like sacrificial melt of portions of the implant body at various locations along the implant. Alternatively, the system can continuously heat and soften the implant body 110B or a combination of softening, melting of cutting the implant body is possible. The system can be use to melt a thermoplastic implant wherein the material retains a very high viscosity, and even a low temperature in comparison to a conventional bone cement, which prevents extravasation. Bone cement 130 can be introduced into the bone as well (FIG. 5).

The embodiments of FIGS. 2A and 5 above described implant bodies 110A and 110B that are transformed to a yielding configuration via resistive heating or Rf ohmic heating of the implant. However, in another system 100A′ an energy source 150B for applying thermal energy to heat the implant and optionally bone tissue can include at least one of an Rf source, a resistive heat source, a light energy source, a microwave source, an ultrasound source, a magnetic source, as shown schematically in FIG. 2B. The energy source 150B can apply energy to the implant via an energy emitter 140B. The material of the implant can carry any biocompatible material that is responsive to a particular energy source such as a chromophore, a ferromagnetic material or the like. Thermal energy application to the implant can transform the implant body into a compliant configuration, melt at least portions of the implant body, sever or cut the implant body, soften and make flexible at least portions of the implant body, or sacrifice portions of an inflexible implant. The thermal energy emitter is disposed at any suitable location in the introducer sleeve 105.

In another embodiment, an implant delivery system 100A″ can include a cryogenic source 150C capable of fragmenting or fracturing at least portions of the implant body. For example a Freon spray can be directed at the implant 110A at a location 140C to weaken, freeze and fracture the distal end of an implant wherein further driving of the implant through the introducer will cause the injection of fragments of the implant body.

In summary, the method of transforming the implant body from unyielding to yielding can utilize at least one of thermal energy application, mechanical energy application and cryogenic cooling to the implant body.

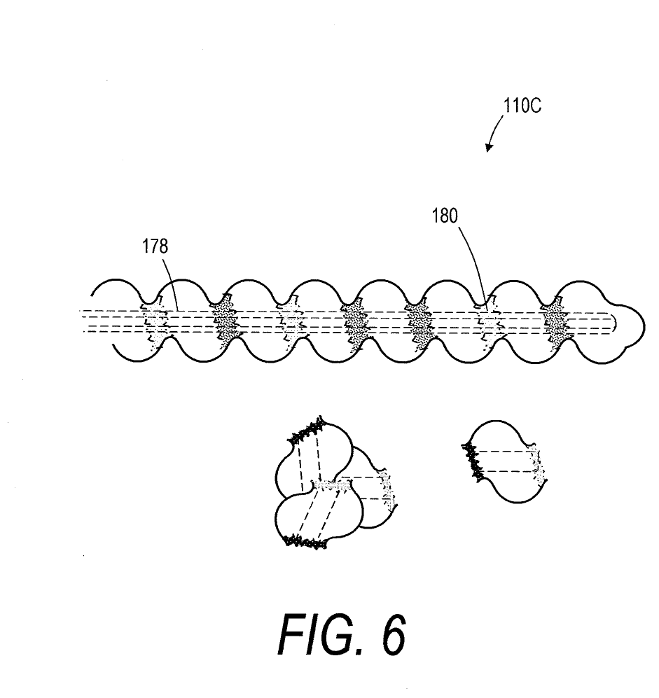

FIG. 6 shows another embodiment of implant body 110C that again has a helical configuration for driving through an introducer sleeve 105 similar to FIGS. 4 and 5. Again, the implant can be driven by a hex rod extending through a bore 178 in the implant. In this embodiment, the implant has spaced apart sacrificial or softenable portions 180 that are configured to fracture, melt, dissolve, or fragment upon thermal energy application, mechanical energy application, chemical application and/or cryogenic cooling to a targeted portion 180 of the implant body. In one embodiment, mechanical force can be applied to implant 110C at the distal end 125 of an introducer sleeve by a bend in the bore 108 of the introducer sleeve 105 as in the side outlet 122 of FIG. 2A.

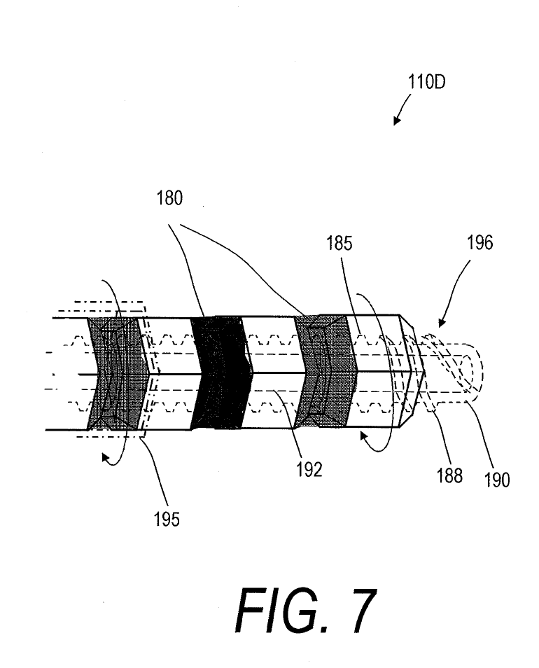

FIG. 7 shows a similar embodiment of implant body 110D that differs in that the helical features 185 are within an interior bore of the implant body that cooperates with threads 188 on shaft 190. The shaft has bore 192 therein that can be used for bone cement delivery. The implant body 110D in this case is driven by an outer sleeve 195 having a polygonal surface that cooperates with a similar surface of the implant body. In this embodiment, the sacrificial or softenable portions 180 are spaced apart and adapted to fracture mechanically by a change in thread pitch in region 196.

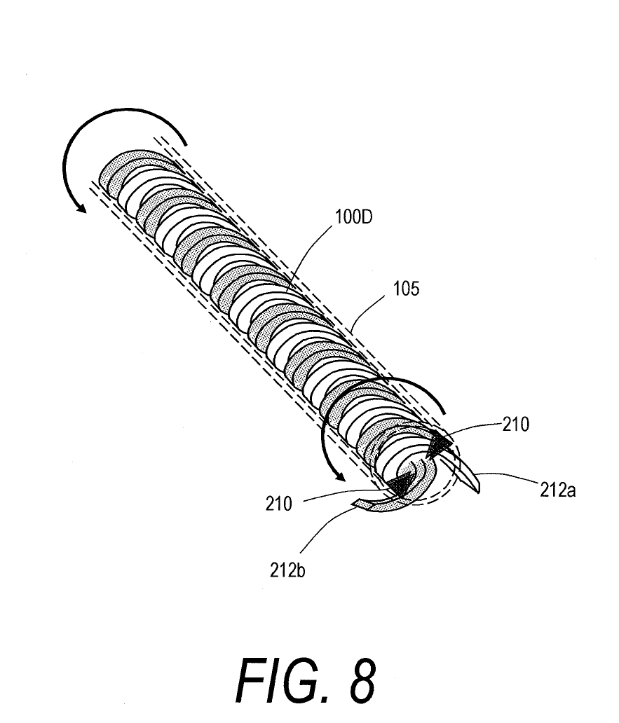

FIG. 8 shows another embodiment of implant body 110E with helical features 205 that are again adapted to cooperate with threads in an interior bore 108 of a sleeve 105 as in FIG. 4. In this embodiment, the sleeve 105 (phantom view) carries blades 210 that are adapted to cut the implant body into flexible strips 212a and 212b for packing into a bone. The implant is again driven by a polygonal rod that engages a central bore in the implant body as described above. The implant body can have a weakened plane about where it is to be cut mechanically.

In any of the embodiments of FIGS. 6, 7 and 8, the implant body can be any biocompatible metal with the fracturable portion being any suitable material such as a polymer.

In any of the embodiments of FIGS. 2A-8, the implant body can be include or comprise a radiopaque composition.

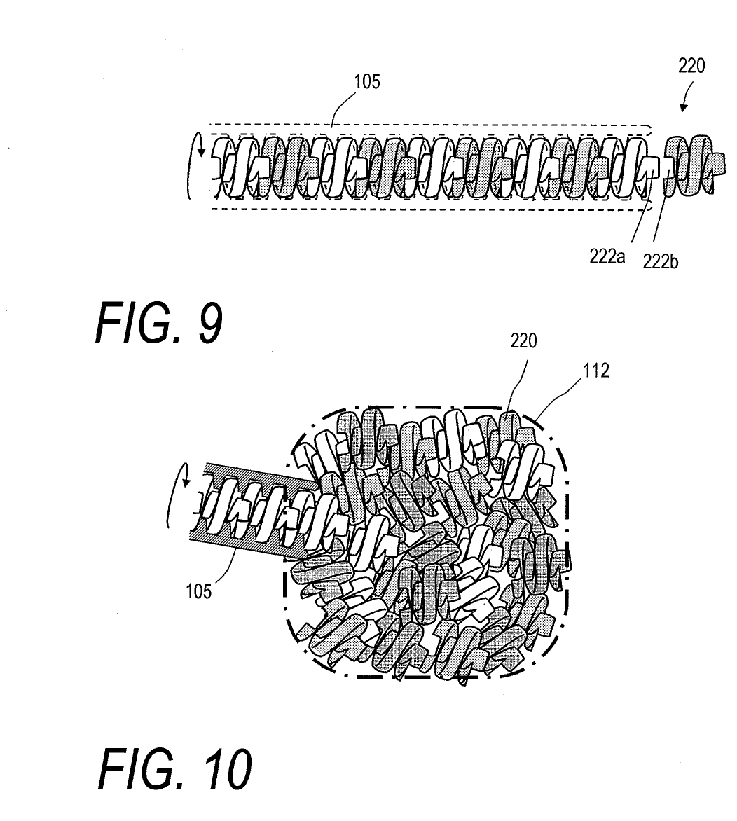

FIGS. 9 and 10 illustrate another embodiment of the invention wherein a plurality of implant elements 220 have helical features 222 that again are adapted to cooperate with threads in an interior bore 108 of a sleeve 105 (cf. FIG. 4). In this embodiment, the implant elements 220 have cooperating key features 222a and 222b to allow cooperative rotation of the assembly for advancement through the sleeve 105. FIG. 10 illustrates a schematic view of a plurality of the elements in a targeted site 112. In another similar embodiment, the elements can be short metal helically formed wires that look a bit like springs that co-operate with a thread feature in at least a distal portion of an introducer sleeve. Such metal wire forms can have a wire feature or molded insert that cooperates with a polygonal driver for helically driving the elements.

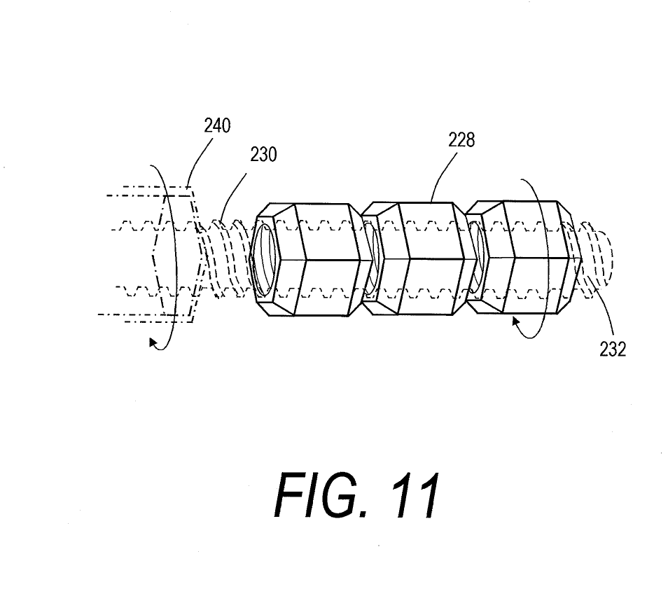

FIG. 11 illustrates another embodiment of the invention wherein a plurality of implant elements 228 have interior helical features that are adapted to cooperate with threads 230 on shaft 232 and the elements 228 are driven by a polygonal outer sleeve 240.

The above description is intended to be illustrative and not exhaustive. Particular characteristics, features, dimensions and the like that are presented in dependent claims can be combined and fall within the scope of the invention. The invention also encompasses embodiments as if dependent claims were alternatively written in a multiple dependent claim format with reference to other independent claims. Specific characteristics and features of the invention and its method are described in relation to some figures and not in others, and this is for convenience only. While the principles of the invention have been made clear in the above descriptions and combinations, it will be obvious to those skilled in the art that modifications may be utilized in the practice of the invention, and otherwise, which are particularly adapted to specific environments and operative requirements without departing from the principles of the invention. The appended claims are intended to cover and embrace any and all such modifications, with the limits only of the true purview, spirit and scope of the invention.

Other features and methods that may be incorporated with the above embodiments may be found in U.S. patent application Ser. No. 11/165,652, filed Jun. 24, 2005; U.S. patent application Ser. No. 11/165,651, filed Jun. 24, 2005, U.S. patent application Ser. No. 11/208,448, filed Aug. 20, 2005; U.S. patent application Ser. No. 11/469,764, filed Sep. 1, 2006; and U.S. application Ser. No. 11/209,035, filed Aug. 22, 2005; and U.S. application Ser. No. 11/196,045, filed Aug. 2, 2005, the entirety of each of which is hereby incorporated by reference.

Of course, the foregoing description is that of certain features, aspects and advantages of the present invention, to which various changes and modifications can be made without departing from the spirit and scope of the present invention. Moreover, the bone treatment systems and methods need not feature all of the objects, advantages, features and aspects discussed above. Thus, for example, those skill in the art will recognize that the invention can be embodied or carried out in a manner that achieves or optimizes one advantage or a group of advantages as taught herein without necessarily achieving other objects or advantages as may be taught or suggested herein. In addition, while a number of variations of the invention have been shown and described in detail, other modifications and methods of use, which are within the scope of this invention, will be readily apparent to those of skill in the art based upon this disclosure. It is contemplated that various combinations or subcombinations of these specific features and aspects of embodiments may be made and still fall within the scope of the invention. Accordingly, it should be understood that various features and aspects of the disclosed embodiments can be combined with or substituted for one another in order to form varying modes of the discussed bone treatment systems and methods.

What is claimed is:

1. A system for treating a bone, comprising:
an elongated introducer configured for insertion in a bone, the introducer defining a passage extending therethrough along an axis of the introducer;
an elongated implant body configured for insertion through the passage into the bone, the elongated implant body having a substantially unyielding configuration; and
an energy source coupled to the elongated introducer, the energy source configured to transform at least a portion of the elongated implant into a yielding configuration to infill the bone.

2. The system of claim 1, wherein the energy source is at least one of an Rf energy source, a resistive heat source, a light energy source, a microwave energy source, an ultrasound energy source, a magnetic energy source, a mechanical energy source and a cryogenic source.

3. The system of claim 2, wherein the energy source is configured to cause at least one of softening, melting, cutting, sacrificing and fracturing said at least one portion of the implant body.

4. The system of claim 1, wherein the energy source comprises at least one energy emitter disposed proximate a distal end of the introducer, the at least one emitter configured to direct thermal energy to at least a portion of the elongated implant.

5. The system of claim 2, wherein the mechanical source comprises an implant drive mechanism and an opening formed on a side of the elongated introducer, said opening configured to fracture at least a portion of the elongated implant as the implant is advanced through the introducer.

6. The system of claim 2, wherein the mechanical source comprises an implant drive mechanism and at least one cutting element coupled to the introducer, said at least one cutting element configured to cut at least a portion of the elongated implant as the implant is advanced through the introducer.

7. The system of claim 1, wherein the energy source is a cryogenic source configured to be coupled to the introducer, the cryogenic source configured to fragment at least a portion of the elongated implant as the implant is advanced through the introducer.

8. The system of claim 1, further comprising a controller in communication with the energy source, the controller configured to control the application of energy by the energy source to at least a portion of the elongated implant.

9. The system of claim 1, wherein the elongated implant body has a helical configuration, the implant body configured to be helically driven through the introducer.

10. The system of claim 9, wherein the elongated implant body comprises a plurality of implant elements, wherein adjacent implant elements are coupled to each other via keys on said adjacent elements.

11. The system of claim 1, wherein the elongated implant body comprises a plurality of helically intertwined portions.

12. The system of claim 1, wherein the elongated implant body comprises a plurality of implant elements, wherein adjacent implant elements are coupled with each other via a sacrificial portion, said energy source configured to effect at least one of fracturing, melting, dissolving and fragmenting of said sacrificial portion.

13. The system of claim 1, wherein the elongated implant body defines a passage therethrough configured to allow a flow of bone fill material therethrough for introduction into the bone.

14. A system for treating a vertebra, comprising:
an elongated introducer configured for insertion in a vertebral body, the introducer defining a passage extending therethrough along an axis of the introducer;
a substantially rigid implant configured for insertion through the passage into the vertebral body, the implant comprising a plurality of implant elements coupled with each other via a junction, the junction transformable between a substantially unyielding configuration to a substantially yielding configuration; and
an energy source coupled to the elongated introducer, the energy source configured to separate at least one implant element from the implant at said junction, the separated implant element advanced into the vertebral body to infill the vertebral body.

15. The system of claim 14, wherein the energy source is at least one of an Rf energy source, a resistive heat source, a light energy source, a microwave energy source, an ultrasound energy source, a magnetic energy source, a mechanical energy source and a cryogenic source.

16. The system of claim 15, wherein the energy source is configured to direct thermal energy to at least said junction to melt said junction to separate the implant element from the substantially rigid implant.

17. The system of claim 14, wherein the energy source is a mechanical source comprising an implant drive mechanism and a member configured to effect at least one of cutting, fracturing and sacrificing of said junction to separate said implant element from the substantially rigid implant.

18. The system of claim 14, wherein the substantially rigid implant has a helical configuration.

19. The system of claim 14, wherein the substantially rigid implant defines a passage therethrough along an axis of the substantially rigid implant, said passage dimensioned to allow a flow of bone fill material therethrough for insertion into the vertebral body.

20. A system for the infill of the interior of a bone, comprising:
an elongated introducer configured for introduction into a bone, the introducer defining a passage extending therethrough along an axis of the introducer;
a plurality of implants dimensioned for advancement along the introducer, each adjacent pair of the plurality of implants coupled to each other via a junction, each implant having a first helical feature for cooperating with a second helical feature on the introducer to helically advance the implants through the passage and into the bone.

21. The system of claim 20, wherein the implants are keyed with one another.

22. The system of claim 20, wherein the implants are coupled to one another via a sacrificial portion.

23. The system of claim 22 further comprising an energy source configured to detach at least one of the plurality of implants proximate said sacrificial portion.

24. A system for treating a bone, comprising:
an elongated introducer configured for insertion into a bone, the introducer defining a passage extending therethrough along an axis of the introducer;
an elongated implant body configured for insertion into the bone along said introducer, the elongated implant body having a substantially unyielding configuration, a surface of the implant body configured to engage with a surface of the introducer for advancement of the implant body along the introducer; and
means for transforming at least a portion of the elongated implant from a substantially unyielding configuration to a yielding configuration to infill the bone.

25. An implant configured for insertion into a bone, comprising:
an elongated body sized for introduction along an introducer into a bone, at least a portion of the elongated body being transformable from a substantially unyielding configuration to a yielding configuration configured for infilling the bone.

26. The implant of claim 25, wherein the elongated body comprises at least one of a polymeric material, a ceramic material, a glass material or a combination thereof.

27. The implant of claim 26, wherein the material is biocompatible.

28. The implant of claim 26, wherein the material comprises PMMA.

29. The implant of claim 25, wherein the elongated body defines a passage therethrough along an axis thereof, the passage configured for insertion of bone cement therethrough into the bone.

30. The implant of claim 25, wherein the elongated body has a helical feature configured to engage a corresponding feature on the introducer.

31. The implant of claim 30, wherein the helical feature is formed on an outer surface of the elongated body.

32. The implant of claim 30, wherein the helical feature is formed on an inner surface of the elongated body.

33. The implant of claim 25, wherein the elongated body comprises a plurality of implant elements and adjacent implant elements are coupled to each other via a junction.

34. The implant of claim 33, wherein the junction is a sacrificial junction.

35. The implant of claim 33, wherein at least one element is severable from an adjacent element proximate said junction via the application of at least one of thermal energy, mechanical energy and cryogenic cooling.

36. The implant of claim 35, wherein thermal energy is applied by at least one of an Rf source, a resistive heat source, a light energy source, a microwave source, an ultrasound source, a magnetic source and a cryogenic source.

37. The implant of claim 33, wherein the junction comprises interlocking key features formed on adjacent implant elements.

38. An implant configured for insertion into a bone, comprising:
an elongated body having at least one helical feature and sized for introduction into a bone, at least a portion of the elongated body being severable from the elongated portion for infilling the bone.

39. The implant of claim 38, wherein the elongated body comprises a plurality of implant elements, adjacent implant elements coupled to each other via a junction.

40. The implant of claim 39, wherein the junction comprises interlocking key features formed on adjacent implant elements.

41. The implant of claim 39, wherein the junction comprises a sacrificial portion.

42. The implant of claim 38, wherein the elongated body defines a passage therethrough along an axis thereof, the passage configured for insertion of bone cement therethrough into the bone.

43. The implant of claim 38, wherein the at least one helical feature is formed on an outer surface of the elongated body, the helical feature configured for engagement with a corresponding feature on an introducer through which the elongated body is advanced into the bone.

44. The implant of claim 38, wherein the at least one helical feature is formed on an inner surface of the elongated body, the helical feature configured for engagement with a corresponding feature on a shaft along which the elongated body is advanced into the bone.
